(19) United States
(12) Patent Application Publication
(10) Pub. No.: US 2011/0008817 A1
(43) Pub. Date: Jan. 13, 2011

(54) MICROFLUIDIC DEVICE HAVING A FLOW CHANNEL WITHIN A GAIN MEDIUM

(76) Inventor: Gary P. Durack, Urbana, IL (US)

Correspondence Address: Woodard, Emhardt, Moriarty, McNett & Henry LLP;Sony Corporation, 111 Monument Circle, Suite 3700, Indianapolis, IN, 46204-5137, US

(21) Appl. No.: 12/830,860

(22) Filed: Jul. 6, 2010

Related U.S. Application Data
(60) Provisional application No. 61/223,728, filed on Jul. 8, 2009.

(51) Int. Cl. C12Q 1/02; C12M 1/34

(52) U.S. Cl. 435/29; 435/288.7

(57) ABSTRACT

The present disclosure relates to microfluidic devices incorporating a gain medium, such as a laser gain medium, and methods for their use. Certain embodiments make use of mirrors integrated into the microfluidic device substrate. Other embodiments are also disclosed.

### CROSS-REFERENCE TO RELATED APPLICATIONS

The present application claims the benefit of U.S. Provisional Patent Application No. 61/223,728, which was filed Jul. 8, 2009 and is hereby incorporated herein by reference in its entirety.

### TECHNICAL FIELD OF THE DISCLOSURE

The present disclosure relates generally to microfluidic cytometry systems and, more particularly, to a microfluidic device having a flow channel within a gain medium.

### BACKGROUND OF THE INVENTION

Flow cytometry-based cell sorting was first introduced to the research community more than 20 years ago. It is a technology that has been widely applied in many areas of life science research, serving as a critical tool for those working in fields such as genetics, immunology, molecular biology and environmental science. Unlike bulk cell separation techniques such as immuno-panning or magnetic column separation, flow cytometry-based cell sorting instruments measure, classify and then sort individual cells or particles serially at rates of several thousand cells per second or higher. This rapid “one-by-one” processing of single cells has made flow cytometry a unique and valuable tool for extracting highly pure sub-populations of cells from otherwise heterogeneous cell suspensions.

Cells targeted for sorting are usually labeled in some manner with a fluorescent material. The fluorescent probes bound to a cell emit fluorescent light as the cell passes through a tightly focused, high intensity, light beam (typically a laser beam). A computer records emission intensities for each cell. These data are then used to classify each cell for specific sorting operations. Flow cytometry-based cell sorting has been successfully applied to hundreds of cell types, cell constituents and microorganisms, as well as many types of inorganic particles of comparable size.

Flow cytometers are also applied widely for rapidly analyzing heterogeneous cell suspensions to identify constituent sub-populations. Examples of the many applications where flow cytometry cell sorting is finding use include isolation of rare populations of immune system cells for AIDS research, isolation of genetically atypical cells for cancer research, isolation of specific chromosomes for genetic studies, and isolation of various species of microorganisms for environmental studies. For example, fluorescently labeled monoclonal antibodies are often used as “markers” to identify immune cells such as T lymphocytes and B lymphocytes, clinical laboratories routinely use this technology to count the number of “CD4 positive” T cells in HIV infected patients, and they also use this technology to identify cells associated with a variety of leukemia and lymphoma cancers.

Recently, two areas of interest are moving cell sorting towards clinical, patient care applications, rather than strictly research applications. First is the move away from chemical pharmaceutical development to the development of biopharmaceuticals. For example, the majority of new cancer therapies are bio-based.

These include a class of antibody-based cancer therapeutics. Cytometry-based cell sorters can play a vital role in the identification, development, purification and, ultimately, production of these products.

Related to this is a move toward the use of cell replacement therapy for patient care. Much of the current interest in stem cells revolves around a new area of medicine often referred to as regenerative therapy or regenerative medicine. These therapies may often require that large numbers of relatively rare cells be isolated from sample patient tissue. For example, adult stem cells may be isolated from bone marrow and ultimately used as part of a re-infusion back into the patient from whom they were removed. Cytometry lends itself very well to such therapies.

There are two basic types of cell sorters in wide use today. They are the “droplet cell sorter” and the “fluid switching cell sorter.” The droplet cell sorter utilizes micro-droplets as containers to transport selected cells to a collection vessel. The micro-droplets are formed by coupling ultrasonic energy to a jetting stream. Droplets containing cells selected for sorting are then electrostatically steered to the desired location. This is a very efficient process, allowing as many as 90,000 cells per second to be sorted from a single stream, limited primarily by the frequency of droplet generation and the time required for illumination.

A detailed description of a prior art flow cytometry system is given in United States Published Patent Application No. US 2005/0112541 A1 to Durack et al.

Droplet cell sorters, however, are not particularly biosafe. Aerosols generated as part of the droplet formation process can carry biohazardous materials. Because of this, biosafe droplet cell sorters have been developed that are contained within a biosafety cabinet so that they may operate within an essentially closed environment. Unfortunately, this type of system does not lend itself to the sterility and operator protection required for routine sorting of patient samples in a clinical environment.

The second type of flow cytometry-based cell sorter is the fluid switching cell sorter. Most fluid switching cell sorters utilize a piezoelectric device to drive a mechanical system which diverts a segment of the flowing sample stream into a collection vessel. Compared to droplet cell sorters, fluid switching cell sorters have a lower maximum cell sorting rate due to the cycle time of the mechanical system used to divert the sample stream. This cycle time, the time between initial sample diversion and when stable non-sorted flow is restored, is typically significantly greater than the period of a droplet generator on a droplet cell sorter. This longer cycle time limits fluid switching cell sorters to processing rates of several hundred cells per second. For the same reason, the stream segment switched by a fluid cell sorter is usually at least ten times the volume of a single micro-drop from a droplet generator. This results in a correspondingly lower concentration of cells in the fluid switching sorter's collection vessel as compared to a droplet sorter's collection vessel.

Newer generation microfluidics technologies offer great promise for improving the efficiency of fluid switching devices and providing cell sorting capability on a chip similar in concept to an electronic integrated circuit. Many microfluidic systems have been demonstrated that can successfully sort cells from heterogeneous cell populations. They have the advantages of being completely self-contained, easy to sterilize, and can be manufactured on sufficient scales (with the resulting manufacturing efficiencies) to be considered a disposable part.

A generic microfluidic device is illustrated in FIG. 1 and indicated generally at 10. The microfluidic device 10 comprises a substrate 12 having a fluid flow channel 14 formed therein by any convenient process as is known in the art. The substrate 12 may be formed from glass, plastic or any other convenient material, and may be substantially transparent or substantially transparent in a portion thereof. The substrate 12 further has three ports 16, 18 and 20 coupled thereto. Port 16 is an inlet port for a sheath fluid. Port 16 has a central axial passage that is in fluid communication with a fluid flow channel 22 that joins fluid flow channel 14 such that sheath fluid entering port 16 from an external supply (not shown) will enter fluid flow channel 22 and then flow into fluid flow channel 14. The sheath fluid supply may be attached to the port 16 by any convenient coupling mechanism as is known to those skilled in the art.

Port 18 also has a central axial passage that is in fluid communication with a fluid flow channel 14 through a sample injection tube 24. Sample injection tube 24 is positioned to be coaxial with the longitudinal axis of the fluid flow channel 14. Injection of a liquid sample of cells into port 18 while sheath fluid is being injected into port 16 will therefore result in the cells flowing through fluid flow channel 14 surrounded by the sheath fluid. The dimensions and configuration of the fluid flow channels 14 and 22, as well as the sample injection tube 24 are chosen so that the sheath/sample fluid will exhibit laminar flow as it travels through the device 10, as is known in the art. Port 20 is coupled to the terminal end of the fluid flow channel 14 so that the sheath/sample fluid may be removed from the microfluidic device 10.

While the sheath/sample fluid is flowing through the fluid flow channel 14, it may be analyzed using cytometry techniques by shining an illumination source through the substrate 12 and into the fluid flow channel 14 at some point between the sample injection tube 24 and the outlet port 20. Additionally, the microfluidic device 10 could be modified to provide for a cell sorting operation, as is known in the art.

Although basic microfluidic devices similar to that described hereinabove have been demonstrated to work well, there is a need in the prior art for improvements to cytometry systems employing microfluidic devices. The present invention is directed to meeting this need.

### SUMMARY OF THE DISCLOSURE

The present disclosure relates to microfluidic devices incorporating a gain medium, such as a laser gain medium, and methods for their use. Certain embodiments make use of mirrors integrated into the microfluidic device substrate. Other embodiments are also disclosed.

In one embodiment, a microfluidic device is disclosed, comprising a substrate, a flow channel formed in said substrate for transport of a liquid sample, and a gain medium formed in said substrate, wherein electromagnetic radiation traversing said gain medium also traverses a portion of said flow channel.

In another embodiment, a microfluidic device is disclosed, comprising a substrate, a flow channel formed in said substrate for transport of a liquid sample, a gain medium formed in said substrate, a first mirror formed in said substrate and disposed on a first side of said gain medium, and a second mirror formed in said substrate and disposed on a second side of said gain medium, wherein electromagnetic radiation reflected between said first and second minors traverses said gain medium and also traverses a portion of said flow channel.

In yet another embodiment, a method of detecting particles in a sample is disclosed, the method comprising the steps of: a) providing a microfluidic device, said microfluidic device comprising: a substrate, a flow channel formed in said substrate for transport of a liquid sample, and a gain medium formed in said substrate, wherein light traversing said gain medium also traverses a portion of said flow channel; b) flowing said sample through said flow channel; c) illuminating said sample with electromagnetic radiation passing through said gain medium and said flow channel and scattering scattered light from said particles; d) performing a cytometry analysis using said scattered light; e) determining a change in radiation output from said gain medium; and f) determining the presence of a particle in the sample based upon said cytometry analysis and said change in radiation output from said gain medium.

In yet another embodiment, a method of detecting particles in a sample is disclosed, the method comprising the steps of: a) flowing a sample through a flow channel; b) passing electromagnetic radiation through a gain medium; c) illuminating said sample with said electromagnetic radiation passed through said gain medium and scattering scattered light from said particles; d) performing a cytometry analysis using said scattered light; e) determining a change in radiation output from said gain medium; and f) determining the presence of a particle in the sample based upon said cytometry analysis and said change in radiation output from said gain medium.

Other embodiments are also disclosed.

### DESCRIPTION OF THE DRAWINGS

FIG. 1 is a perspective view of a prior art microfluidic device.

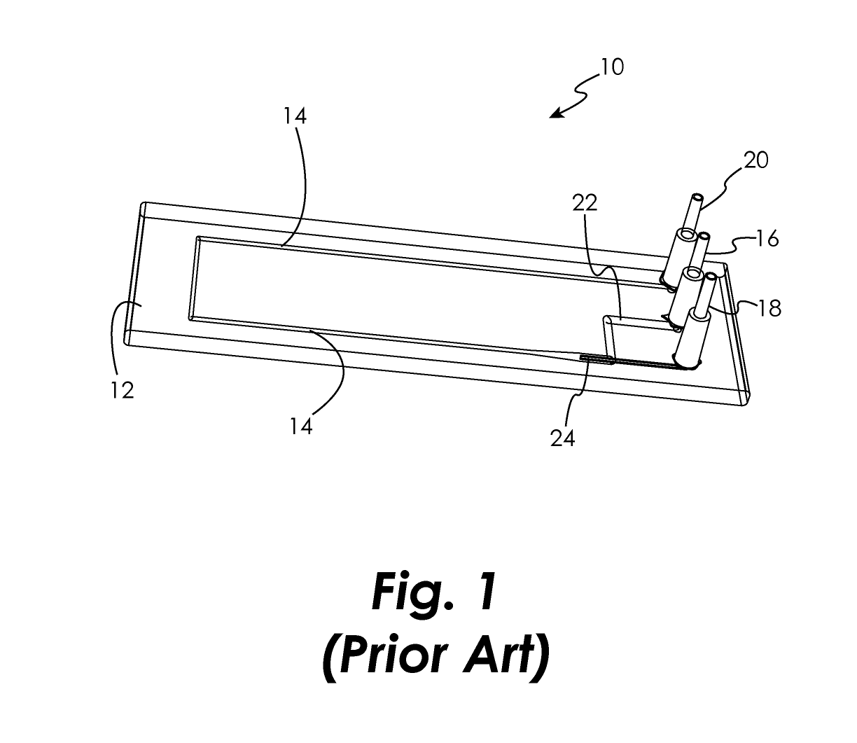

FIG. 2 is a schematic, close-up front view of a flow channel and laser system on a microfluidic device according to an embodiment of the present disclosure.

FIG. 3 is a schematic perspective view of a microfluidic device according to an embodiment of the present disclosure.

FIG. 4 is a schematic perspective view of a microfluidic device according to an embodiment of the present disclosure.

FIG. 5 is a schematic perspective view of a microfluidic device according to an embodiment of the present disclosure.

FIG. 6 is a schematic perspective view of a microfluidic device according to an embodiment of the present disclosure.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein are contemplated as would normally occur to one skilled in the art to which the disclosure relates.

The present disclosure is generally directed to microfluidic devices, such as cytometry chips, having flow channels positioned within a gain medium of an optical cavity or optical resonator. In certain embodiments, positioning a cytometry flow channel within a gain medium provides the researcher or medical professional with increased ability to detect small particles traveling through the channel. The particles traveling through the cytometry flow channel will interact with the electromagnetic radiation (for example, infrared, ultraviolet or visible light, to name just a few non-limiting examples) traveling through the gain medium and the channel in such a manner that characteristics and parameters (such as optical gain) of the optical cavity or optical resonator are modified by the presence of particular fluids or particles in the flow channel. The modification of the characteristics or parameters of the optical cavity or optical resonator caused by the interaction with the fluid or particles (or a single particle) can then be measured by monitoring time dependent changes in the radiation output from the resonator, such as intensity, wavelength, linewidth, or polarization.

Laser technologies often involve the use of optical resonators or optical cavities that consist of oppositely aligned mirrors with a gain medium positioned between the mirrors. The gain medium functions to multiply photons traveling therethrough between the mirrors. The photons reflect off the minors and continually multiply through the gain medium, the number of photons being a function of the path through the gain medium. The gain medium can be any appropriate gain medium as would occur to one of ordinary skill in the art, such as a solid state material having electrons, a dye, and an ionized gas. Other non-limiting examples of laser gain media include:
Certain crystals, typically doped with rare-earth ions (for example neodymium, ytterbium, or erbium) or transition metal ions (titanium or chromium); most often yttrium aluminium garnet (YAG), yttrium orthovanadate (YVO₄), or sapphire (Al₂O₃);
Glasses, for example silicate or phosphate glasses, doped with laser-active ions;
Gases, for example mixtures of helium and neon (HeNe), nitrogen, argon, carbon monoxide, carbon dioxide, or metal vapors;
Semiconductors, for example gallium arsenide (GaAs), indium gallium arsenide (InGaAs), or gallium nitride (GaN).

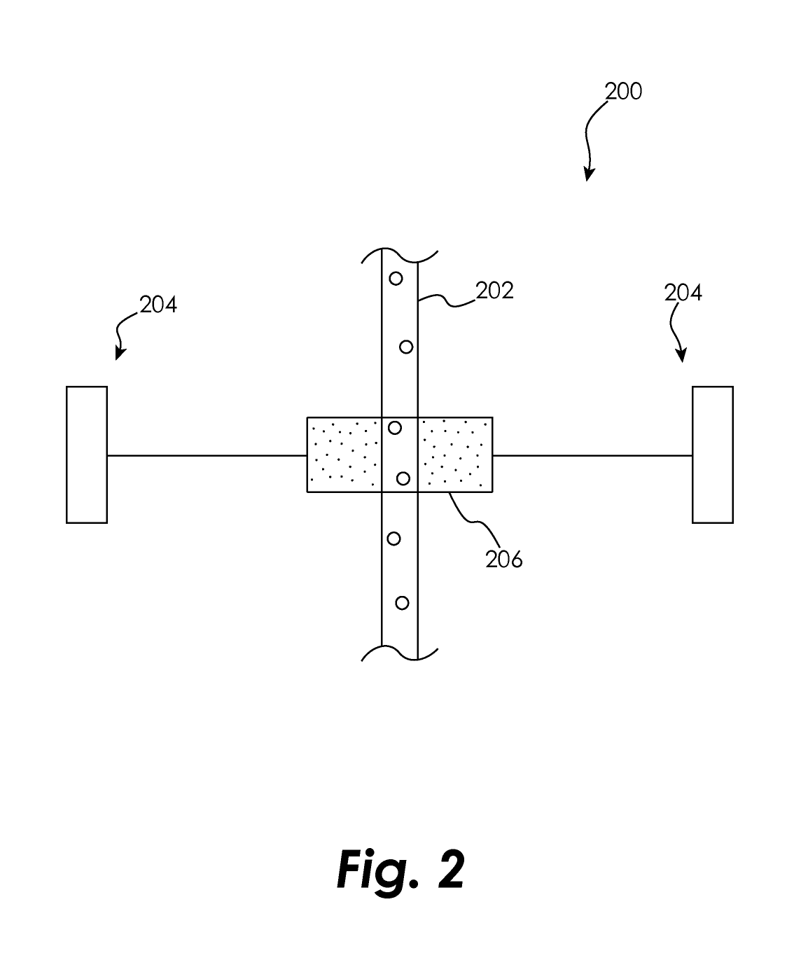

FIG. 2 schematically illustrates a system 200 having an example cytometry flow channel 202 extending through a section of gain medium 206 (such as a laser gain medium, for example) on a microfluidic device as part of a cytometry analysis occurring with respect to the device (the specific cytometry analysis operations are not critical to the present disclosure). In certain embodiments the gain medium 206 is formed as a first portion on a first side of flow channel 202 and a second portion on a second side of flow channel 202. In other embodiments, the gain medium 206 completely surrounds a portion of the flow channel 202. As illustrated, two minors 204 are positioned on opposite sides of a gain medium 206 to reflect laser photons back and forth through the gain medium 206, thus forming an optical cavity or optical resonator with the flow channel integral to it. In such embodiments, the photons will multiply every time they travel through gain medium 206, creating optic gain for the cavity or laser system. As particles travel through the flow channel 202 and reach the section of channel 202 surrounded by gain medium 206, the particles will intereact with some of the photons passing between the mirrors. The particles may scatter or absorb some of the light traveling through the gain medium 206. Depending on the molecules present in or on the particles, additional photons may be emitted due to fluorescence or scattering processes. As a result, the observable characteristics of the optical cavity or resonator will change in a manner corresponding to the interaction of the particle with the radiation passing through the gain medium. The modification of the characteristics or parameters of the optical cavity or optical resonator caused by the interaction with the fluid or particles (or a single particle) can then be measured by monitoring time dependent changes in the radiation output from the resonator, such as intensity, wavelength, linewidth, or polarization. For example, the optical gain of the cavity or resonator will be reduced when photons that would have otherwise passed through the gain medium to the opposing minor are scattered or absorbed. Based on that reduction of optic gain, measurable by monitoring the output of the optical cavity or the power within the cavity, as well as the scattering of light (detected by the cytometry analysis), the presence of a particle in the flow channel 202 can be detected. It should be appreciated that the flow channel 202, gain medium 206, and minors 204 can be shaped, sized, positioned and configured differently as would occur to one of ordinary skill in the art. Scattering of light by the particles may include fluorescence, Raman scatter, phosphorescence, luminescence, or scatter, just to name a few non-limiting examples.

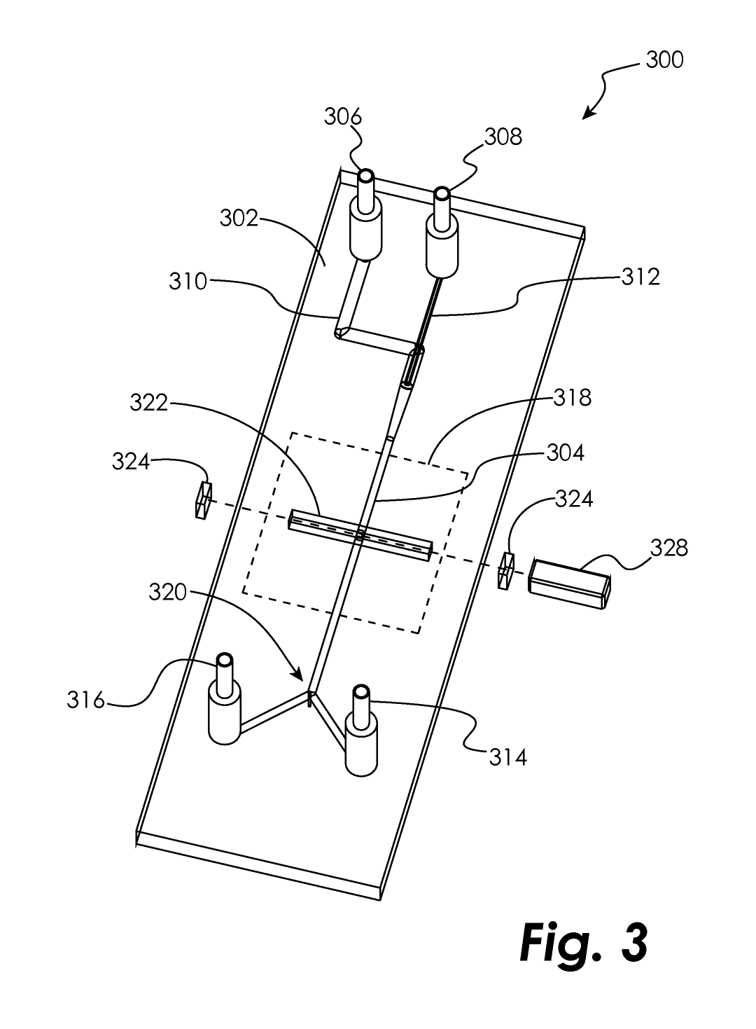

Referring now to FIG. 3, a microfluidic device is schematically illustrated and indicated generally at 300. The microfluidic device 300 comprises a substrate 302 having a fluid flow channel 304 formed therein by any convenient process as is known in the art. The substrate 302 may be formed from glass, plastic or any other convenient material, and may be substantially transparent or substantially transparent in a portion thereof. The substrate 302 further has two inlet ports 306 and 308 coupled thereto. Port 306 is an inlet port for a sheath fluid. Port 306 has a central axial passage that is in fluid communication with a fluid flow channel 310 that joins fluid flow channel 304 such that sheath fluid entering port 306 from an external supply (not shown) will enter fluid flow channel 304 and then flow into fluid flow channel 304. The sheath fluid supply may be attached to the port 306 by any convenient coupling mechanism as is known to those skilled in the art.

Port 308 also has a central axial passage that is in fluid communication with fluid flow channel 304 through a sample injection tube 312. Sample injection tube 312 is positioned to be coaxial with the longitudinal axis of the fluid flow channel 304. Injection of a liquid sample of cells into port 308 while sheath fluid is being injected into port 306 will therefore result in the cells flowing through fluid flow channel 304 surrounded by the sheath fluid. The dimensions and configuration of the fluid flow channels 304 and 310, as well as the sample injection tube 312 are chosen so that the sheath/sample fluid will exhibit laminar flow as it travels through the device 300, as is known in the art.

The substrate 302 further has two outlet ports 314 and 316 coupled thereto. As described in greater detail hereinbelow, the sample flowing through flow channel 304 may be sorted using cytometry techniques. Sample that is identified as being desirable is directed to collection port 314, while the remainder of the fluid sample is directed to waste port 316.

While the sheath/sample fluid is flowing through the fluid flow channel 304, it may be analyzed using cytometry techniques by shining an illumination source through the substrate 302 and into the fluid flow channel 304 at some point between the sample injection tube 312 and the outlet ports 314 and 316, such as cytometry analysis area 318. Based upon the results of the cytometry analysis performed in area 318, desired sample fluid may be diverted to outlet port 314 by appropriate control of flow diverter 320. Similarly, undesired cells in the sample may be diverted to the waste port 316 by appropriate control of flow diverter 320.

In one embodiment, the flow diverter 320 is a piezoelectric device that can be actuated with an electric command signal in order to mechanically divert the flow through the sorting channel 304 into either the outlet port 314 or the waste port 316, depending upon the position of the flow diverter 320. In other embodiments, flow diverter 320 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

In order to facilitate the cytometry analysis performed in area 318, the cytometry flow channel 304 extends through a section of laser gain medium 322 in the substrate 302. In certain embodiments the laser gain medium 322 is formed as a first portion on a first side of flow channel 304 and a second portion on a second side of flow channel 304. In other embodiments, the laser gain medium 322 completely surrounds a portion of the flow channel 304. Two minors 324 are positioned on opposite sides of the gain medium 322 to reflect laser photons back and forth through the gain medium 322, thus forming an optical cavity or optical resonator. As is typical for the case of a laser, one of the minors 324, often referred to as an output coupler, is less reflective than the opposing mirror. This allows radiation to be emitted from the cavity or resonator. The mirrors and other optics in the cavity are designed in a typical manner known to those skilled in the art to allow the laser 328 to pass through the gain medium which includes the integral flow channel 304. The laser 328 injects radiation into the gain medium and serves as a “pump” for the emission captured within the cavity. With sufficient energy density appropriate for the optical resonator, lasing will occur within the cavity. There are many examples of such designs such as diode pumped solid state and dye lasers. This pumping can also be supplied directly with electrical current, as is the case with diode lasers or by electrical discharge as is the case with gas ion lasers. As discussed hereinabove, the photons with characteristics consistent with the cavity or resonator design [only certain wavelengths and polarization are supported by a specific cavity based on the properties of the minors and the electromagnetic radiation modes (transverse and longitudinal) supported by the cavity length and mirror design.] will multiply every time they travel through gain medium 322, creating optical gain for the laser system. As particles travel through the flow channel 304 and reach the section of channel 304 surrounded by gain medium 322, the particles will interact with some of the photons and will scatter some of the light traveling through the gain medium 322. Depending on the molecules present in or on the particles, additional photons may be emitted due to fluorescence or scattering processes. As a result, the observable characteristics of the optical cavity or resonator will change in a manner corresponding to the interaction of the particle with the radiation passing through the gain medium. For example the optical gain of the cavity or resonator will be reduced when photons that would have otherwise passed through the gain medium to the opposing mirror are scattered or absorbed. Based on that reduction of optic gain, measurable by monitoring the output of the optical cavity or the power within the cavity, as well as the scattering of light (detected by the cytometry analysis), the presence of a particle in the flow channel can be detected and characteristics of the particle (such as size) may be determined. Once detected, flow diverter 320 may be controlled to direct the sample portion to the appropriate outlet port 314, 316. It should be appreciated that the flow channel 304, gain medium 322, and minors 324 can be shaped, sized, positioned and configured differently as would occur to one of ordinary skill in the art.

With all of the embodiments disclosed herein, the use of a microfluidic device on a substrate offers many advantages, one of which is that the microfluidic device may be treated as a disposable part, allowing a new microfluidic device to be used for sorting each new sample of cells. This greatly simplifies the handling of the sorting equipment and reduces the complexity of cleaning the equipment to prevent cross contamination between sorting sessions, because much of the hardware through which the samples flow is simply disposed of. The microfluidic device also lends itself well to sterilization (such as by gamma irradiation) before being disposed of.

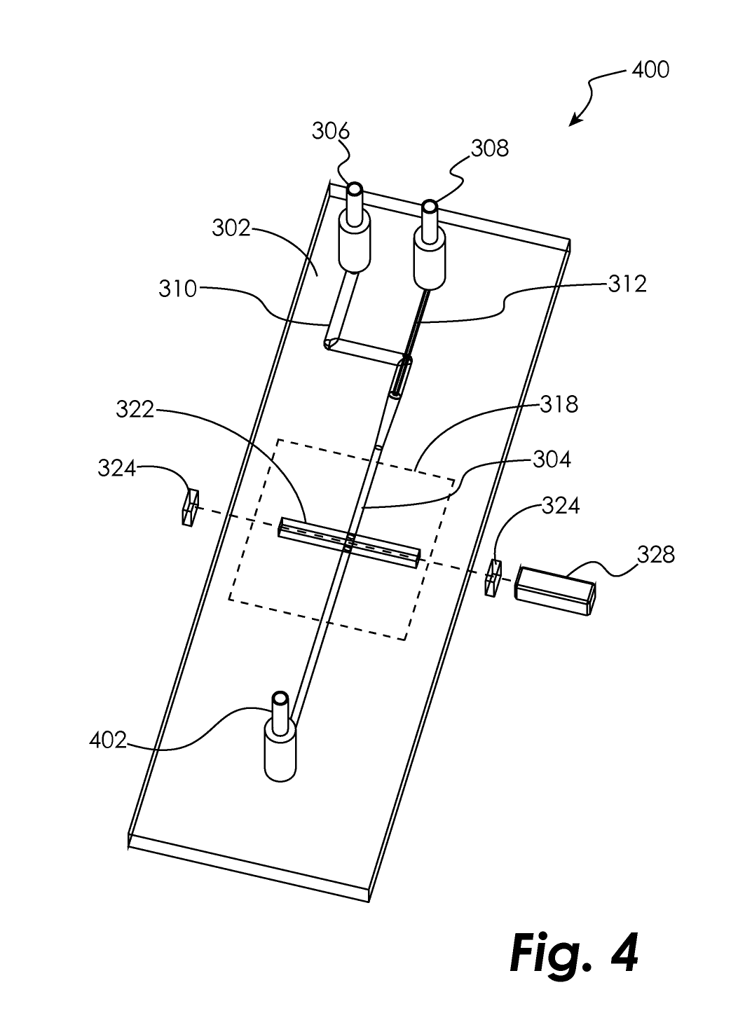

In certain embodiments, the sample fluid flowing in flow channel 304 is not sorted on the microfluidic chip. As schematically illustrated in FIG. 4 and indicated generally at 400, the microfluidic device 400 is similar to the microfluidic device 300 and like reference designators are used for like portions. When using the microfluidic device 400, the cytometry analysis and particle detection derived from the output of the laser cavity (eg. reduction in laser gain) are used to detect the presence of a particle in the sample fluid flowing through flow channel 304 for the purpose of counting the number of such particles present within the sample, rather than for the purpose of sorting the sample onboard the microfluidic device. Therefore, once the sample has passed through the cytometry analysis section 318, the entire sample is routed to outlet port 402.

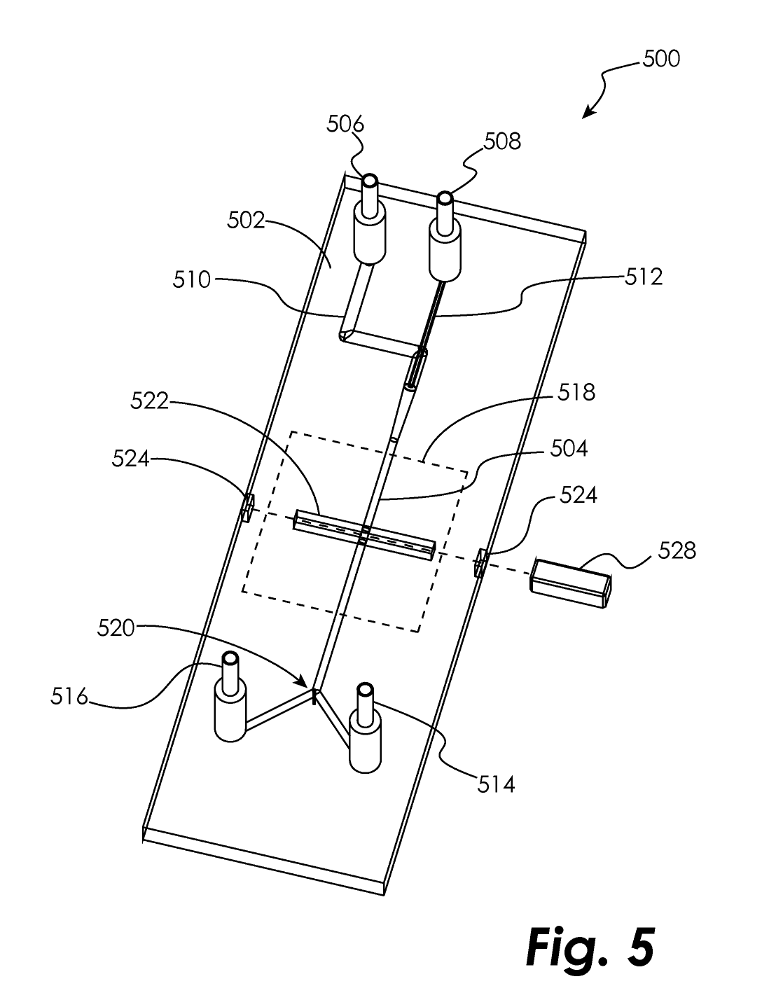

Referring now to FIG. 5, another embodiment of a microfluidic device is schematically illustrated and indicated generally at 500. The microfluidic device 500 comprises a substrate 502 having a fluid flow channel 504 formed therein by any convenient process as is known in the art. The substrate 502 may be formed from glass, plastic or any other convenient material, and may be substantially transparent or substantially transparent in a portion thereof. The substrate 502 further has two inlet ports 506 and 508 coupled thereto. Port 506 is an inlet port for a sheath fluid. Port 506 has a central axial passage that is in fluid communication with a fluid flow channel 510 that joins fluid flow channel 504 such that sheath fluid entering port 506 from an external supply (not shown) will enter fluid flow channel 504 and then flow into fluid flow channel 504. The sheath fluid supply may be attached to the port 506 by any convenient coupling mechanism as is known to those skilled in the art.

Port 508 also has a central axial passage that is in fluid communication with fluid flow channel 504 through a sample injection tube 512. Sample injection tube 512 is positioned to be coaxial with the longitudinal axis of the fluid flow channel 504. Injection of a liquid sample of cells into port 508 while sheath fluid is being injected into port 506 will therefore result in the cells flowing through fluid flow channel 504 surrounded by the sheath fluid. The dimensions and configuration of the fluid flow channels 504 and 510, as well as the sample injection tube 512 are chosen so that the sheath/sample fluid will exhibit a defined flow as it travels through the device 500, as is known in the art.

The substrate 502 further has two outlet ports 514 and 516 coupled thereto. As described in greater detail hereinbelow, the sample flowing through flow channel 504 may be sorted using cytometry techniques. Sample that is identified as being desirable is directed to collection port 514, while the remainder of the fluid sample is directed to waste port 516.

While the sheath/sample fluid is flowing through the fluid flow channel 504, it may be analyzed using cytometry techniques by shining an illumination source through the substrate 502 and into the fluid flow channel 504 at some point between the sample injection tube 512 and the outlet ports 514 and 516, such as cytometry analysis area 518. Based upon the results of the cytometry analysis performed in area 518, desired sample fluid may be diverted to outlet port 514 by appropriate control of flow diverter 520. Similarly, undesired cells in the sample may be diverted to the waste port 516 by appropriate control of flow diverter 520.

In order to facilitate the cytometry analysis performed in area 518, the cytometry flow channel 504 extends through a section of laser gain medium 522 in the substrate 502. In certain embodiments the laser gain medium 522 is formed as a first portion on a first side of flow channel 504 and a second portion on a second side of flow channel 504. In other embodiments, the laser gain medium 522 completely surrounds a portion of the flow channel 504. Two minors 524 formed integrally with substrate 502 or affixed thereto are positioned on opposite sides of the gain medium 522 to reflect laser photons back and forth through the gain medium 522, thus forming an optical cavity or optical resonator. As is typical for the case of a laser, one of the minors 524, often referred to as an output coupler, is less reflective than the opposing minor. This allows radiation to be emitted from the cavity or resonator. The minors and other optics in the cavity are designed in a typical manner known to those skilled in the art to allow the laser 528 to pass through the gain medium which includes the integral flow channel 504. The laser 528 injects radiation into the gain medium and serves as a “pump” for the emission captured within the cavity. With sufficient energy density appropriate for the optical resonator, lasing will occur within the cavity. There are many examples of such designs such as diode pumped solid state and dye lasers. This pumping can also be supplied directly with electrical current, as is the case with diode lasers or by electrical discharge as is the case with gas ion lasers. As discussed hereinabove, the photons with characteristics consistent with the cavity or resonator design [only certain wavelengths and polarization are supported by a specific cavity based on the properties of the minors and the electromagnetic radiation modes (transverse and longitudinal) supported by the cavity length and minor design.] will multiply every time they travel through gain medium 522, creating optical gain for the laser system. As particles travel through the flow channel 504 and reach the section of channel 504 surrounded by gain medium 522, the particles will interact with some of the photons and will scatter some of the light traveling through the gain medium 522. Depending on the molecules present in or on the particles, additional photons may be emitted due to fluorescence or scattering processes. As a result, the observable characteristics of the optical cavity or resonator will change in a manner corresponding to the interaction of the particle with the radiation passing through the gain medium. For example the optical gain of the cavity or resonator will be reduced when photons that would have otherwise passed through the gain medium to the opposing mirror are scattered or absorbed. Based on that reduction of optic gain, measurable by monitoring the output of the optical cavity or the power within the cavity, as well as the scattering of light (detected by the cytometry analysis), the presence of a particle in the flow channel can be detected and characteristics of the particle (such as size) may be determined. Once detected, flow diverter 520 may be controlled to direct the sample portion to the appropriate outlet port 514, 516. It should be appreciated that the flow channel 504, gain medium 522, and mirrors 524 can be shaped, sized, positioned and configured differently as would occur to one of ordinary skill in the art.

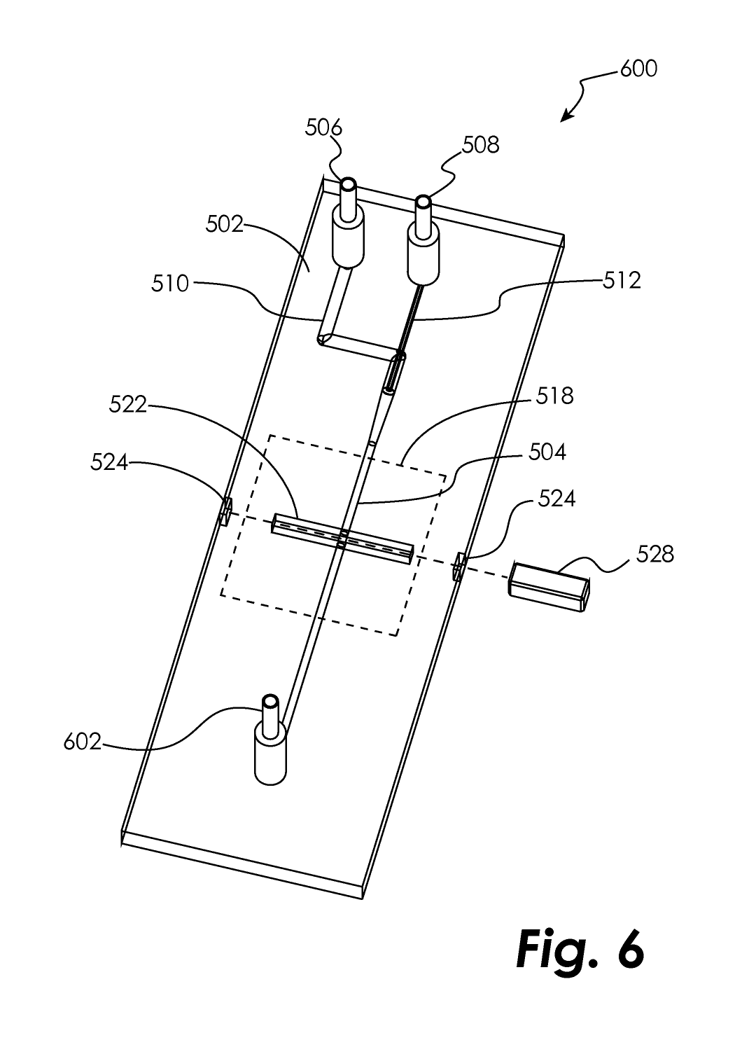

In certain embodiments, the sample fluid flowing in flow channel 504 is not sorted on the microfluidic chip. As schematically illustrated in FIG. 6 and indicated generally at 600, the microfluidic device 600 is similar to the microfluidic device 500 and like reference designators are used for like portions. When using the microfluidic device 600, the cytometry analysis and particle detection derived from the output of the laser cavity (e.g. reduction in laser gain) are used to detect the presence of a particle in the sample fluid flowing through flow channel 504 for the purpose of counting the number of such particles present within the sample, rather than for the purpose of sorting the sample onboard the microfluidic device. Therefore, once the sample has passed through the cytometry analysis section 518, all of the sample is routed to outlet port 602.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the disclosure are desired to be protected.

What is claimed:

1. A microfluidic device, comprising:
a substrate;
a flow channel formed in said substrate for transport of a liquid sample; and
a gain medium formed in said substrate;
wherein electromagnetic radiation traversing said gain medium also traverses a portion of said flow channel.

2. The microfluidic device of claim 1, wherein said gain medium comprises a laser gain medium and said electromagnetic radiation comprises light.

3. The microfluidic device of claim 1, wherein a first portion of said gain medium is disposed on a first side of said flow channel and a second portion of said gain medium is disposed on a second side of said flow channel.

4. The microfluidic device of claim 1, wherein a portion of said gain medium surrounds a portion of said flow channel.

5. The microfluidic device of claim 1, further comprising:
a first minor formed in said substrate and disposed on a first side of said gain medium; and
a second mirror formed in said substrate and disposed on a second side of said gain medium

6. The microfluidic device of claim 5, where said first and second mirrors are arranged such that an optical cavity is formed where the electromagnetic radiation contained in the optical cavity interacts with the flow channel.

7. The microfluidic device of claim 6, wherein said optical cavity comprises an optical resonator.

8. The microfluidic device of claim 5, wherein said first and second mirrors comprise minors selected from the group consisting of: convex, concave, planar, compound surfaces, and combinations thereof.

9. The microfluidic device of claim 1, further comprising:
a source of sheath fluid coupled to said flow channel; and
a source of analyte sample coupled to said flow channel.

10. The microfluidic device of claim 1, further comprising:
a sorted sample channel formed in said substrate;
a waste channel formed in said substrate;
a flow diverter having a flow diverter input coupled to said flow channel, a first flow diverter outlet coupled to said sorted sample channel, and a second flow diverter outlet coupled to said waste channel.

11. The microfluidic device of claim 10, wherein said flow diverter is selected from the group consisting of: piezoelectric devices, air bubble insertion means, and magnetically actuated fluid deflectors.

12. The microfluidic device of claim 1, further comprising an output port coupled to said flow channel.

13. A microfluidic device, comprising:
a substrate;
a flow channel formed in said substrate for transport of a liquid sample;
a gain medium formed in said substrate;
a first mirror formed in said substrate and disposed on a first side of said gain medium; and
a second minor formed in said substrate and disposed on a second side of said gain medium;
wherein electromagnetic radiation reflected between said first and second mirrors traverses said gain medium and also traverses a portion of said flow channel.

14. The microfluidic device of claim 13, wherein said first and second minors comprise minors selected from the group consisting of: convex, concave, planar, compound surfaces, and combinations thereof.

15. The microfluidic device of claim 13, wherein said gain medium comprises a laser gain medium and said electromagnetic radiation comprises light.

16. The microfluidic device of claim 13, wherein a first portion of said gain medium is disposed on a first side of said flow channel and a second portion of said gain medium is disposed on a second side of said flow channel.

17. The microfluidic device of claim 13, wherein a portion of said gain medium surrounds a portion of said flow channel.

18. The microfluidic device of claim 13, further comprising:
a source of sheath fluid coupled to said flow channel; and
a source of analyte sample coupled to said flow channel.

19. The microfluidic device of claim 13, further comprising:
a sorted sample channel formed in said substrate;
a waste channel formed in said substrate;
a flow diverter having a flow diverter input coupled to said flow channel, a first flow diverter outlet coupled to said sorted sample channel, and a second flow diverter outlet coupled to said waste channel.

20. The microfluidic device of claim 19, wherein said flow diverter is selected from the group consisting of: piezoelectric devices, air bubble insertion means, and magnetically actuated fluid deflectors.

21. The microfluidic device of claim 13, further comprising an output port coupled to said flow channel.

22. A method of detecting particles in a sample, the method comprising the steps of:
a) providing a microfluidic device, said microfluidic device comprising:
a substrate;
a flow channel formed in said substrate for transport of a liquid sample; and
a gain medium formed in said substrate;
wherein light traversing said gain medium also traverses a portion of said flow channel;
b) flowing said sample through said flow channel;
c) illuminating said sample with electromagnetic radiation passing through said gain medium and said flow channel and scattering scattered light from said particles;
d) performing a cytometry analysis using said scattered light;
e) determining a change in radiation output from said gain medium; and
f) determining the presence of a particle in the sample based upon said cytometry analysis and said change in radiation output from said gain medium.

23. The method of claim 22, wherein step (e) comprises monitoring time dependent changes in the radiation output from said gain medium.

24. The method of claim 23, wherein time dependent changes in the radiation output from said gain medium is selected from the group consisting of:
intensity, wavelength, linewidth, or polarization.

25. The method of claim 24, wherein said gain medium comprises a laser gain medium and said electromagnetic radiation comprises light.

26. The method of claim 22, further comprising the step of:
g) sorting said sample based upon the determination made at step (f).

27. The method of claim 22, further comprising the steps of:
g) directing said sample to a first destination if said determination made at step (f) indicates that a particle is present; and
h) directing said sample to a second destination if said determination made at step (f) indicates that no particle is present.

28. The method of claim 22, further comprising the step of:
g) diverting flow in said flow channel based upon the determination made at step (f).

29. The method of claim 28, wherein step (g) comprises an action selected from the group consisting of: actuating a piezoelectric device, inserting an air bubble into said respective flow channel, and magnetically actuating a fluid deflector.

30. The method of claim 22, wherein said sample comprises biological cells.

31. The method of claim 22, further comprising the steps of:
g) sterilizing the microfluidic device; and
h) disposing of the microfluidic device.

32. The method of claim 22, wherein said scattering is selected from the group consisting of: fluorescent emission, Raman scatter, phosphorescence, and luminescence.

33. A method of detecting particles in a sample, the method comprising the steps of:
a) flowing a sample through a flow channel;
b) passing electromagnetic radiation through a gain medium;
c) illuminating said sample with said electromagnetic radiation passed through said gain medium and scattering scattered light from said particles;
d) performing a cytometry analysis using said scattered light;
e) determining a change in radiation output from said gain medium; and
f) determining the presence of a particle in the sample based upon said cytometry analysis and said change in radiation output from said gain medium.

34. The method of claim 33, wherein step (e) comprises monitoring time dependent changes in the radiation output from said gain medium.

35. The method of claim 34, wherein time dependent changes in the radiation output from said gain medium is selected from the group consisting of:
intensity, wavelength, linewidth, or polarization.

36. The method of claim 33, wherein said gain medium comprises a laser gain medium and said electromagnetic radiation comprises light.

37. The method of claim 33, further comprising the step of:
g) sorting said sample based upon the determination made at step (f).

38. The method of claim 33, further comprising the steps of:
g) directing said sample to a first destination if said determination made at step (f) indicates that a particle is present; and
h) directing said sample to a second destination if said determination made at step (f) indicates that no particle is present.

39. The method of claim 33, further comprising the step of:
g) diverting flow in said flow channel based upon the determination made at step (f).

40. The method of claim 39, wherein step (g) comprises an action selected from the group consisting of: actuating a piezoelectric device, inserting an air bubble into said respective flow channel, and magnetically actuating a fluid deflector.

41. The method of claim 33, wherein said particles comprise biological cells.

42. The method of claim 33, wherein said flow channel is in a microfluidic device, the method further comprising the steps of:
g) sterilizing the microfluidic device; and
h) disposing of the microfluidic device.

43. The method of claim 33, wherein said scattering is selected from the group consisting of: fluorescent emission, Raman scatter, phosphorescence, and luminescence.
